(19) United States
(12) Patent Application Publication
(10) Pub. No.: US 2007/0225565 A1
(43) Pub. Date: Sep. 27, 2007

(54) INSERTION PORTION OF AN ENDOSCOPE

(75) Inventor: Takayuki OGINO, Saitama (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/688,374

(22) Filed: Mar. 20, 2007

(30) Foreign Application Priority Data
Mar. 22, 2006 (JP) 2006-077915

(51) Int. Cl. A61B 1/00

(52) U.S. Cl. 600/142; 600/153

(57) ABSTRACT

An insertion portion of an endoscope includes a flexible tubular framework composed of short-cylindrical joint rings coupled in series via rotatable connecting shafts; and flexible internal elements including a treatment tool insertion channel and a optical fiber bundle(s). Each short-cylindrical joint ring is coupled at one end to one adjacent joint ring via two connecting shafts at 180-degree symmetrical positions and is coupled at the other end to another adjacent joint ring via another two connecting shafts at 180-degree symmetrical positions with a 90-degree phase shift with respect to the one adjacent joint ring. Inner end surfaces of one set of connecting shafts at a specific circumferential position are arranged to face the treatment tool insertion channel, and inner end surfaces of another set of the connecting shafts at another specific circumferential direction are arranged to face or substantially face the optical fiber bundle.

### BACKGROUND OF THE INVENTION

1. Field of the Invention

The present invention relates to an insertion portion of an endoscope.

2. Description of the Related Art

An insertion portion of an endoscope which is introduced into a body is generally constructed of a metal helical tube, a mesh tube and a flexible jacket, wherein the metal helical tube is sheathed with the mesh tube and further sheathed with the flexible jacket. However, in the case where the endoscope is sterilized by a process of steaming under pressure (autoclaving), the metal helical tube may shrink. Accordingly, to achieve durability against autoclaving, the helical tube may be replaced by a flexible tubular framework consisting of a plurality of short-cylindrical joint rings which are coupled in series via connecting shafts to be freely rotatable (as shown in, e.g., Japanese unexamined patent publication H09-24020).

In the aforementioned flexible tubular framework that is used as a replacement for a helical tube, each joint ring is coupled at one end thereof to one adjacent joint ring to be freely rotatable relative thereto via two connecting shafts at 180-degree symmetrical positions, respectively, and is further coupled at the other end thereof to another adjacent joint ring to be freely rotatable relative thereto via another two connecting shafts at 180-degree symmetrical positions, respectively, with a 90-degree phase shift with respect to the aforementioned one adjacent joint ring

Due to this configuration, the inner end surfaces (radially inner end surfaces) of four connecting shafts 92 that connect one joint ring 91 to two adjacent joint rings 91 project radially inwards at equi-angular intervals (intervals of 90 degrees) from the inner peripheral surface of the flexible tubular framework as shown in FIG. 5. Accordingly, internal elements which have a large cross-sectional area such as a treatment tool insertion channel 93 and two optical fiber bundles 94 for lighting are inserted into the flexible tubular framework to be installed therein to be positioned in between the connecting shafts 92 (i.e., at positions deviating from the connecting shafts 92 at an approximately 45 degrees).

However, if the flexible tubular framework having the aforementioned type of structure, which is constructed of the plurality of joint rings 91 which are coupled in series through the connecting shafts 92, is fully bent to the maximum, the flexible tubular framework bends largely at a great angle (i.e., at a small radius of curvature) in a direction which deviates from the position of the axis of each pair of radially-opposed connecting shafts 92 at an angle of 45 degrees. In this direction, which deviates from the position of the axis of each pair of radially-opposed connecting shafts 92 at an angle of 45 degrees, the flexible tubular framework can bend largely because the maximum bend angles of the two bending directions of the flexible tubular framework (i.e., two directions deviating from each other at an angle of 90 degrees) are combined.

Therefore, when the flexible tubular framework is fully bent to the maximum, the treatment tool insertion channel 93, which is greater in diameter than an air/water supply tube, etc., and tends to buckle easily when bent, and the two optical bundle fibers 94, whose optical fibers break strand by strand if bent repeatedly at a small radius of curvature, are brought to the innermost position at the bent portion (curved portion) of the flexible tubular framework, which causes the treatment tool insertion channel 93 and the two optical bundle fibers 94 to be damaged within a short period of time, and accordingly, there is a possibility of the endoscope itself being rendered inoperable within a short period of time.

### SUMMARY OF THE INVENTION

The present invention provides an insertion portion of an endoscope which uses a flexible tubular framework instead of a helical tube, wherein the flexible tubular framework is composed of a plurality of joint rings which are coupled in series via connecting shafts to be freely rotatable, and wherein one or more treatment tool insertion channels and one or more illumination optical fiber bundles among the various internal elements inserted into the flexible tubular framework to be installed therein, which easily damage by bending, can obtain excellent durability.

According to an aspect of the present invention, an insertion portion of an endoscope is provided, including a bendable flexible tubular framework composed of a plurality of short-cylindrical joint rings which are coupled in series via rotatable connecting shafts; and a plurality of flexible internal elements including at least one treatment tool insertion channel and at least one optical fiber bundle for lighting which are inserted into the flexible tubular framework. Each of the plurality of short-cylindrical joint rings is coupled at one end thereof to one adjacent joint ring of the plurality of joint rings via two connecting shafts of the connecting shafts at 180-degree symmetrical positions, respectively, and is coupled at the other end thereof to another adjacent joint ring of the plurality of joint rings via another two connecting shafts of the connecting shafts at 180-degree symmetrical positions, respectively, with a 90-degree phase shift with respect to the one adjacent joint ring. Inner end surfaces of one set of the connecting shafts which are positioned at a specific circumferential position and are aligned in an axial direction of the bendable flexible tubular framework are arranged to face the treatment tool insertion channel. Inner end surfaces of another set of the connecting shafts which are positioned at another specific circumferential position and are aligned in an axial direction of the bendable flexible tubular framework are arranged to one of face and substantially face the optical fiber bundle.

It is desirable for the connecting shafts to include a first pair of radially-opposed connecting shafts provided on each of the plurality of joint rings; and a second pair of radially-opposed connecting shafts provided on the each of the plurality of joint rings, a common axis of the first pair of radially-opposed connecting shafts extending at a different angle relative to a common axis of the second pair of radially-opposed connecting shafts as viewed in an axial direction of the each of the plurality of joint rings. The first pair of radially-opposed connecting shafts, provided on the plurality of joint rings, are aligned in a direction orthogonal to an axial direction of the tubular framework, and the second pairs of radially-opposed connecting shafts, provided on the plurality of joint rings, are aligned in the orthogonal direction.

It is desirable for the one set of connecting shafts to be positioned on a first straight line passing through an axis of the flexible tubular framework and lying on an orthogonal axial plane of the treatment tool insertion channel, and for the another set of connecting shafts is positioned on, or in a close vicinity of, a second straight line passing through the axis of the flexible tubular framework and lying on an orthogonal axial plane of the optical fiber bundle. It is desirable for the inner end surfaces of the adjacent connecting shafts and the inner end surfaces of the another adjacent connecting shafts to project radially inwards from an inner peripheral surface of the flexible tubular framework.

It is desirable for the plurality of flexible internal elements to further include at least one flexible tube for fluid transmission and at least one electric signal cable.

According to the present invention, the treatment tool insertion channel and the optical fiber bundle among various internal elements inserted into the flexible tubular framework to be installed therein, which are easily damaged by bending, are resistant to buckling and breaking, thus achieving excellent durability due to the configuration in which each of at least one treatment tool insertion channel and at least one optical fiber bundle is positioned at one of a position facing the inner end surface of one adjacent connecting shaft in the axial direction thereof and a position substantially facing the inner end surface of another adjacent connecting shaft in the insertion portion of the endoscope which uses a flexible tubular framework instead of a helical tube, wherein the flexible tubular framework is composed of a plurality of joint rings which are coupled in series via connecting shafts to be freely rotatable.

The present disclosure relates to subject matter contained in Japanese Patent Application No. 2006-77915 (filed on Mar. 22, 2006) which is expressly incorporated herein by reference in its entirety.

### BRIEF DESCRIPTION OF THE DRAWINGS

The present invention will be discussed below in detail with reference to the accompanying drawings, in which:

FIG. 1 is a cross sectional view of an insertion portion of an embodiment of an endoscope according to the present invention, taken along a plane orthogonal to the axis of the insertion portion flexible tube (taken along I-I line shown in FIG. 3);

FIG. 2 is a side view of the endoscope according to the present invention, showing the overall structure thereof;

FIG. 3 is a side elevational view, partly in cross section, of the flexible tubular framework of the insertion portion of the endoscope according to the present invention;

FIG. 4 is a perspective view of a joint ring of the flexible tubular framework of the insertion portion of the endoscope according to the present invention; and

FIG. 5 is a cross sectional view of an insertion portion of a conventional endoscope, taken along a plane orthogonal to the axis of the flexible tubular framework of the insertion portion.

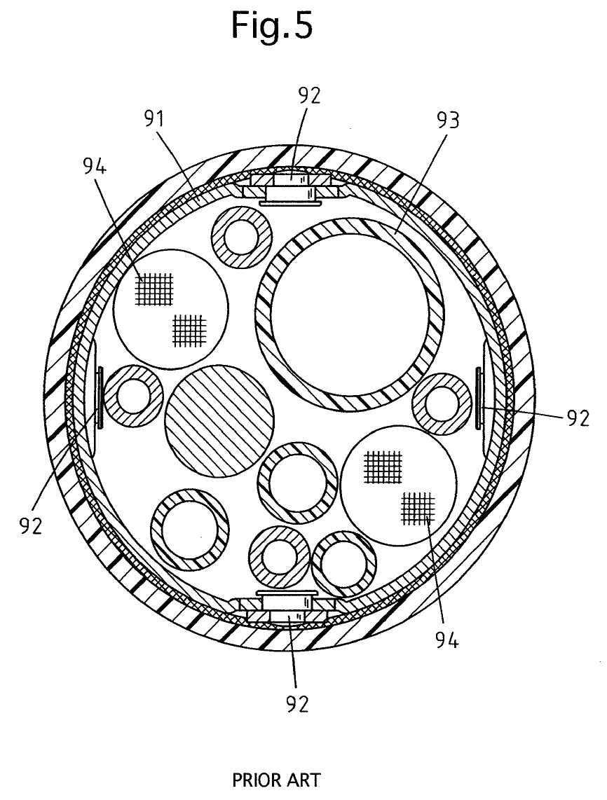

### PREFERRED EMBODIMENT OF THE PRESENT INVENTION

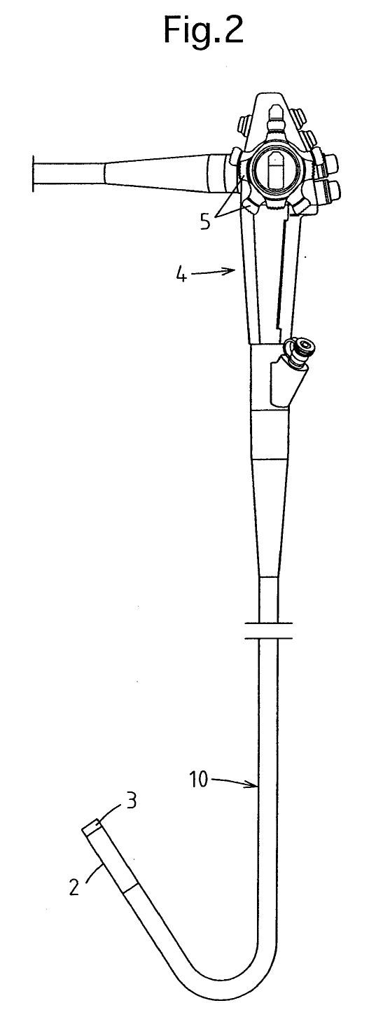

FIG. 2 shows the overall structure of an endoscope 1 according to the present invention. The endoscope 1 is provided with a control body 4 and an insertion portion connected to the control body 4. The distal end portion of the insertion portion is formed as a steerable bendable portion 2. The insertion portion of the endoscope 1 is provided with a flexible tubular portion 10, and the steerable bendable portion 2 is joined to the front end of the flexible tubular portion 10. The insertion portion of the endoscope 1 is further provided at the front end of the steerable bendable portion 2 with an end body 3 in which an objective window (not shown) and other openings/nozzles are provided. The steerable bendable portion 2 can be steered to bend freely (right, left, upward and downward) by controlling steering knobs 5 provided on the control body 4.

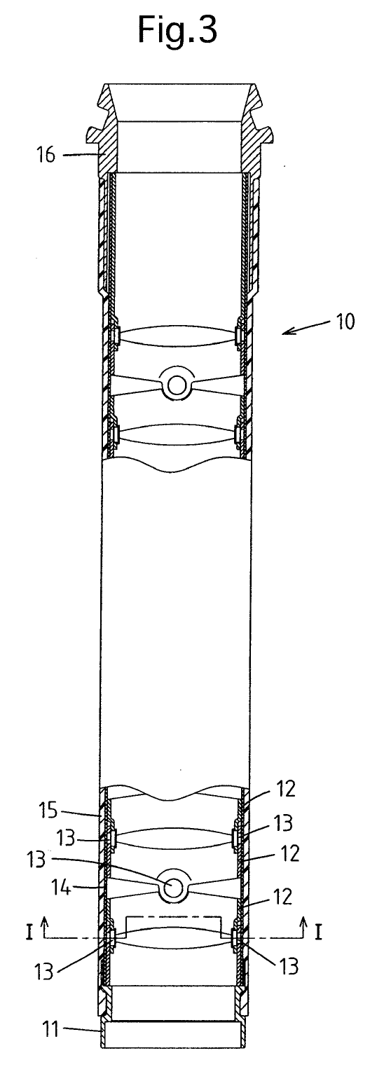

FIG. 3 shows the flexible tubular portion 10. The flexible tubular portion 10 in this embodiment of the endoscope is provided with a freely-bendable flexible tubular framework composed of a plurality (e.g., several dozen to several hundreds) of short-cylindrical joint rings 12 which are coupled in series via connecting shafts 13 to be freely rotatable. The axis of each shaft 13 is orientated to pass through the axis of the associated joint ring 12. For instance, the connecting shafts 13 can be formed as rivets. The outer periphery of the freely-bendable flexible tubular framework is sheathed with a braided sheath (mesh tube) 14 which is formed by braiding fine metal strands, and the outer periphery of the braided sheath 14 is sheathed with a flexible jacket 15.

The flexible tubular portion 10 is provided at a front end (bottom end as viewed in FIG. 3) thereof with a distal-end mouth ring 11 which is formed to be connected to the steerable bendable portion 2. The flexible tubular portion 10 is provided at a proximal end (top end as viewed in FIG. 3) thereof with a proximal-end mouth ring 16 which is formed to be connected to the control body 4. Although various internal elements which will be discussed later are inserted into the flexible tubular portion 10 to be installed therein, such internal elements are not shown in FIG. 3 for the purpose of clarity.

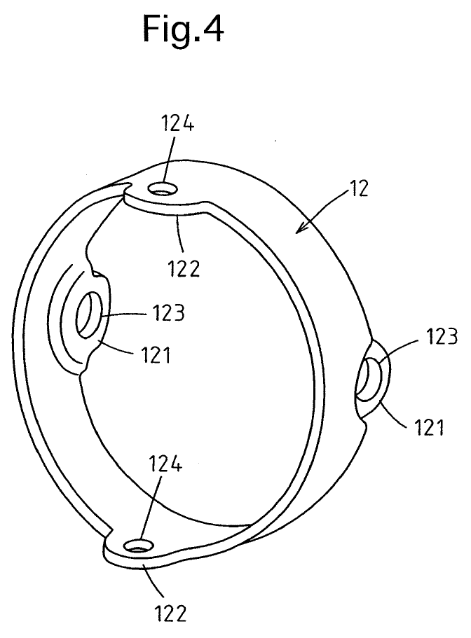

For instance, each of the plurality of short-cylindrical joint rings 12 is formed into as shape as shown in FIG. 4. Namely, the joint ring 12 is provided at one end thereof with a pair of tongues 121 which are positioned symmetrically at 180-degree positions, respectively, and is further provided at the other end thereof with a pair of tongues 122 which are positioned symmetrically at 180-degree positions, respectively, at a 90-degree phase shift with respect to the pair of tongues 121 about the axis of the joint ring 12.

The pair of tongues 121 are formed in a manner such that the radially outer surfaces thereof are each recessed radially inwards by the amount of thickness of each of the pair of tongues 122 so that the pair of tongues 122 of one joint ring 12 are overlaid on the pair of tongues 121 of an adjacent joint ring 12 with the radially outer surfaces of the pair of tongues 122 being substantially flush with the outer peripheral surface of the adjacent joint ring 12 when the plurality of short-cylindrical joint rings 12 are arranged in series so that the axes thereof are aligned. A through-hole 123 through which one connecting shaft 13 passes is made in each tongue 121 and a through-hole 124 through which one connecting shaft 13 passes is made in each tongue 122.

Therefore, each joint ring 12 is coupled at one end thereof to one adjacent joint ring 12 via two connecting shafts (a first pair of radially-opposed connecting shafts) 13 at 180-degree symmetrical positions, respectively, and is further coupled at the other end thereof to another adjacent joint ring 12 via another two connecting shafts (a second pair of radially-opposed connecting shafts) 13 at 180-degree symmetrical positions, respectively, with a 90-degree phase shift with respect to the aforementioned one adjacent joint ring 12. Accordingly, the first pair of radially-opposed connecting shafts 13 are aligned and the common axis thereof passes through the axis of the associated joint ring 12. Likewise, the second pair of radially-opposed connecting shafts 13 are aligned and the common axis thereof passes through the axis of the associated joint ring 12. The first pairs of radially-opposed connecting shafts 13 of the plurality of joint rings 12 connected in series are aligned in the axial direction of the freely-bendable flexible tubular framework, respectively. Likewise, the second pairs of radially-opposed connecting shafts 13 of the plurality of joint rings 12 connected in series are aligned in the axial direction of the freely-bendable flexible tubular framework, respectively.

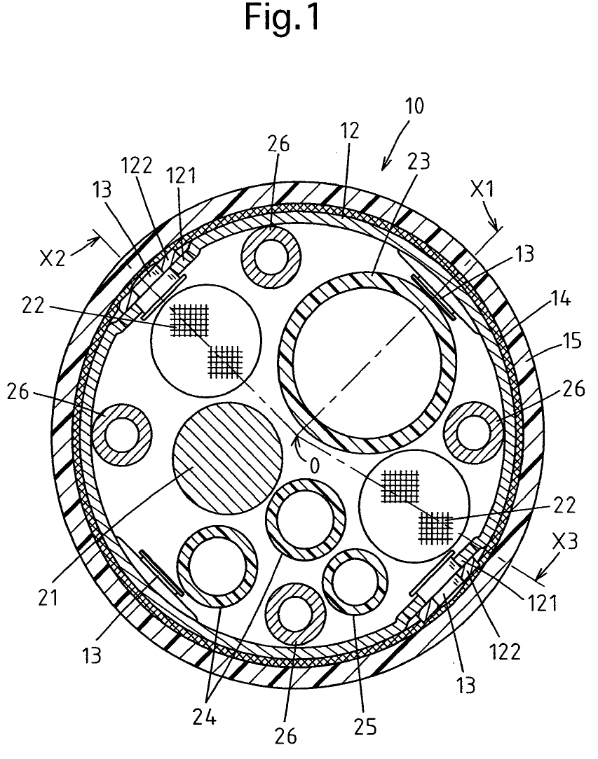

FIG. 1 is a cross sectional view of the flexible tubular portion 10 in which various internal elements (flexible internal elements) have been inserted and installed, taken along I-I line shown in FIG. 3. Such internal elements include an electric signal cable 21 for transmitting signals such as an imaging signal, and two optical fiber bundles 22 for lighting. Each of these internal elements 21 and 22 is sheathed with a silicon-rubber tube or the like.

The endoscope 1 is further provided in the flexible tubular portion 10 with a treatment tool insertion channel 23 made of, e.g., a fluorocarbon resin, into which a treatment tool (not shown) is inserted, two air/water supply tubes 24, each of which is capable of serving as an air supply tube or a water supply tube for blowing air or spraying water on the surface of the aforementioned objective window, a sub-water supply tube 25 for spraying water toward the front of the end body 3, and four guide coils 26 into which four control wires (not shown) that are moved to manipulate the steerable bendable portion 2 by manually operating the steering knobs 5 are inserted to pass through the four guide coils 26, respectively. All these internal elements (21 through 26) have high flexibility.

The electric signal cable 21, the two optical fiber bundles 22, the treatment tool insertion channel 23, the two air/water supply tubes 24 and the sub-water supply tube 25 are fixed at the respective front ends (fixing extreme ends) thereof to the end body 3 and inserted straightly into the flexible tubular portion 10 to run therethrough straightly from these fixed front ends in a direction parallel to the axis of the flexible tubular portion 10.

Each of the four guide coils 26 is fixed to the inner peripheral surface of the front-end mouth ring 11 (or fixed to the inner peripheral surface of a connecting mouth-ring (not shown) via which the flexible tubular portion 10 and the steerable bendable portion 2 are connected to each other) and inserted straight into the flexible tubular portion 10 to run therethrough in a direction parallel to the axis of the flexible tubular portion 10.

Therefore, all the internal elements (21 through 26) are each inserted into the flexible tubular framework (12 and 13) to pass therethrough straightly from the fixing extreme ends of the internal elements (21 through 26). Additionally, as shown in FIG. 1, each of the treatment tool insertion channel 23 and the two optical fiber bundles 22 is positioned at either a position facing the inner end surface of one adjacent connecting shaft 13 in the axial direction thereof or a position substantially facing the inner end surface of connecting shaft 13. Specifically, in the particular embodiment shown in FIG. 1, as viewed in FIG. 1, the upper-left optical fiber bundle 22 and the treatment tool insertion channel 23 are positioned so as to face the upper-left connecting shaft 13 and the upper-right connecting shaft 13 in the axial directions thereof, respectively, while the lower-right optical fiber bundle 22 is positioned so as to substantially face the lower-right connecting shaft 13.

Namely, one of the four connecting shafts 13 (the upper-right connecting shaft 13 as viewed in FIG. 1) that are positioned at intervals of 90 degrees is positioned on a straight line X1 passing through an axis 0 of the flexible tubular framework (12 and 13) (i.e., the axis of the associated joint ring 12) and lying on an orthogonal axial plane of the treatment tool insertion channel 23, another connecting shaft 13 (the upper-left connecting shaft 13 as viewed in FIG. 1) is positioned on a straight line X2 passing through the axis 0 of the flexible tubular framework (12 and 13) and lying on an orthogonal axial plane of one of the two optical fiber bundles 22, and another connecting shaft 13 (the lower-right connecting shaft 13 as viewed in FIG. 1) is positioned in the close vicinity of a straight line X3 passing through the axis 0 of the flexible tubular framework (12 and 13) and lying on an orthogonal axial plane of the other optical fiber bundle 22.

Consequently, in the insertion portion of the endoscope using the aforementioned flexible tubular framework, that is composed of the plurality of joint rings 12 which are coupled in series through the connecting shafts 13 to be freely rotatable, instead of using a helical tube, the treatment tool insertion channel 23 and each optical fiber bundle 22 among various internal elements inserted into the flexible tubular framework to be installed therein, which are easily damaged by bending, become resistant to buckling and breaking, thus achieving excellent durability because none of the treatment tool insertion channel 23 and the two optical fiber bundles 22 are brought to the innermost position at the bent portion (curved portion) of the flexible tubular framework when the flexible tubular portion 10 is fully bent to the maximum in a direction which deviates from the position of the axis of each pair of radially-opposed connecting shafts 13 by an angle of 45 degrees.

Obvious changes may be made in the specific embodiment of the present invention described herein, such modifications being within the spirit and scope of the invention claimed. It is indicated that all matter contained herein is illustrative and does not limit the scope of the present invention.

What is claimed is:

1. An insertion portion of an endoscope comprising:
a bendable flexible tubular framework composed of a plurality of short-cylindrical joint rings which are coupled in series via rotatable connecting shafts; and
a plurality of flexible internal elements including at least one treatment tool insertion channel and at least one optical fiber bundle for lighting which are inserted into said flexible tubular framework;
wherein each of said plurality of short-cylindrical joint rings is coupled at one end thereof to one adjacent joint ring of said plurality of joint rings via two connecting shafts of said connecting shafts at 180-degree symmetrical positions, respectively, and is coupled at the other end thereof to another adjacent joint ring of said plurality of joint rings via another two connecting shafts of said connecting shafts at 180-degree symmetrical positions, respectively, with a 90-degree phase shift with respect to said one adjacent joint ring,
wherein inner end surfaces of one set of said connecting shafts which are positioned at a specific circumferential position and are aligned in an axial direction of said bendable flexible tubular framework are arranged to face said treatment tool insertion channel, and
wherein inner end surfaces of another set of said connecting shafts which are positioned at another specific circumferential position and are aligned in an axial direction of said bendable flexible tubular framework are arranged to one of face and substantially face said optical fiber bundle.

2. The insertion portion of the endoscope according to claim 1, wherein said connecting shafts comprise:
a first pair of radially-opposed connecting shafts provided on each of said plurality of joint rings; and
a second pair of radially-opposed connecting shafts provided on said each of said plurality of joint rings, a common axis of said first pair of radially-opposed connecting shafts extending at a different angle relative to a common axis of said second pair of radially-opposed connecting shafts as viewed in an axial direction of said each of said plurality of joint rings,
wherein said first pair of radially-opposed connecting shafts, provided on said plurality of joint rings, are aligned in a direction orthogonal to an axial direction of said tubular framework, and
wherein said second pairs of radially-opposed connecting shafts, provided on said plurality of joint rings, are aligned in said orthogonal direction.

3. The insertion portion of the endoscope according to claim 1, wherein said one set of connecting shafts is positioned on a first straight line passing through an axis of said flexible tubular framework and lying on an orthogonal axial plane of said treatment tool insertion channel, and
wherein said another set of connecting shafts is positioned on, or in a close vicinity of, a second straight line passing through said axis of said flexible tubular framework and lying on an orthogonal axial plane of said optical fiber bundle.

4. The insertion portion of the endoscope according to claim 1, wherein said inner end surfaces of said adjacent connecting shafts and said inner end surfaces of said another adjacent connecting shafts project radially inwards from an inner peripheral surface of said flexible tubular framework.

5. The insertion portion of the endoscope according to claim 1, wherein said plurality of flexible internal elements further include at least one flexible tube for fluid transmission and at least one electric signal cable.
